(19) United States
(12) Patent Application Publication
(10) Pub. No.: US 2023/0233432 A1
(43) Pub. Date: Jul. 27, 2023

(54) PROCESS FOR THE PREPARATION OF A COMPOSITION OF LIPOAMINO ACIDS AND DIOLS

(71) Applicant: SOCIETE D'EXPLOITATION DE PRODUITS POUR LES INDUSTRIES CHIMIQUES SEPPIC, Paris Cedex 07 (FR)

(72) Inventors: Jérôme GUILBOT, Castres (FR); Georges DACOSTA, Saix (FR); Virginie BARTHE, Paris Cedex 07 (FR)

(21) Appl. No.: 18/185,099

(22) Filed: Mar. 16, 2023

Related U.S. Application Data
(62) Division of application No. 16/956,420, filed on Jun. 19, 2020.

(30) Foreign Application Priority Data
Dec. 20, 2017 (FR) 1762585

(51) Int. Cl. A61K 47/18; A61K 8/44; A61K 8/34; A61K 47/10; A61K 9/00; A61Q 19/00

(52) U.S. Cl. CPC A61K 47/183; A61K 8/44; A61K 8/345; A61K 9/0014; A61K 47/10; A61Q 19/00; A61K 2800/10

(57) ABSTRACT

Disclosed are a new composition of lipoamino acids and diols, a process for the preparation thereof, and a cosmetic or pharmaceutical composition resulting therefrom.

### CROSS REFERENCE TO RELATED APPLICATIONS

This application is a Divisional of Application No. 16/956,420, filed on Jun. 19, 2020, which is the National Phase under 35 U.S.C. § 371 of International Application No. PCT/FR2018/053213, filed on Dec. 12, 2018, which claims the benefit under 35 U.S.C. § 119(a) to Patent Application No. 1762585, filed in France on Dec. 20, 2017, all of which are hereby expressly incorporated by reference into the present application.

### BACKGROUND OF THE INVENTION

### Field of the Invention

The present invention relates to a novel process for the preparation of N-acyl derivatives of amino acids, of (oligo)peptides or else of partial or total protein hydrolysates, having a high degree of conversion of the starting materials used. The present invention also relates to the use of these N-acyl compounds obtained for preparing cosmetic or pharmaceutical compositions for topical use or industrial detergency compositions.

### Description of the Related Art

N-Acyl amino acid derivatives, also known as lipoamino acids (LAAs), are anionic surfactants which are formed by a polar head originating from a residue of at least one amino acid, or else from a residue of (oligo)peptides or else from residues of partial or total protein hydrolysates, and by a hydrocarbon-based chain of lipophilic nature, originating from fatty acid chlorides or fatty acid methyl esters, which are themselves derived from oleochemistry.

These N-acyl derivatives of amino acids, of (oligo)peptides or else of partial or total protein hydrolysates are commonly used first of all as ingredients which contribute foaming and cleansing properties for the preparation of cosmetic compositions, such as, for example, shower gels or shampoos, or else as ingredients which contribute biological properties for the preparation of cosmetic compositions intended to prevent or correct unsightly skin effects; said biological properties are, for example, anti-aging, slimming, firming, depigmenting or pro-pigmenting properties.

The N-acyl derivatives of amino acids, of (oligo)peptides or else of partial or total protein hydrolysates are commonly synthesized by acylation of one or more amino acids in the presence of acid chloride, under experimental conditions known as Schotten-Baumann conditions.

Such a process is disclosed, for example, in the American patents US 2 463 779 and US 6 703 517, in the publication J. Am. Oil Chem. Soc. 78 (1956) 172, and in the international applications published under the numbers WO 92/21318 and WO 94/26694.

This acylation process comprises a prior step of salification of the amino acid, followed by a step of acylation of the amino acid salt with an acid chloride, and then acidification of the N-acyl salt obtained.

The first step consists in neutralizing the amino acid, dissolved beforehand in water or in a mixture of water and an organic cosolvent, with an inorganic base, usually aqueous sodium hydroxide or aqueous potassium hydroxide. The carboxyl group is then in an ionized form, thus allowing better solubility of the amino acid in water. The pH of this aqueous solution is between 9.0 and 12.0, which ensures that the amine function of the amino acid is not protonated.

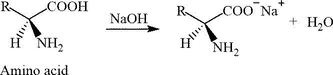

The second step is the acylation step proper. At this stage, the acid chloride is added gradually to the neutralized amino acid solution, at ambient temperature. The nucleophilic amine function attacks the electrophilic carbon of the carbonyl function. This results in the formation of an amide bond between the two starting substrates and also the formation of hydrochloric acid. This acid is directly neutralized in situ by gradual addition of a mineral base (regulation of the pH to about 10.0).

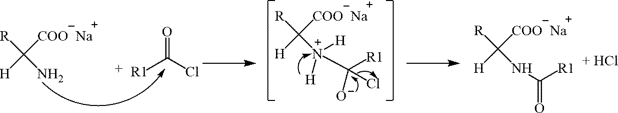

At this stage, the side reaction of hydrolysis of the acid chloride to give a soap is also possible. It must, however, be minimized so as to achieve a satisfactory conversion of the amino acid to its N-acyl derivative and to efficiently isolate them, since an excessively high content of soap (or fatty acid salt) may induce phase separation of the reaction medium and/or odor or toxicity problems (for C₈ and C′₁₁ acyl chains, for example).

The two main reaction parameters that enable the formation of soap to be controlled are the stirring speed during the reaction phase, optimization of which makes possible an improvement in the contact surface area of the chloride with the medium, and the optional addition, during the step of dissolution of the amino acid, of an acylation cosolvent, such as, for example, acetone, methyl ethyl ketone, isopropanol or glycols.

This addition of cosolvent makes it possible to improve the affinity of the acid chloride for the reaction medium. In such a case, the acylation cosolvent must be astutely chosen so as to avoid or minimize the formation of new side products resulting from the reaction between this same cosolvent and the acid chloride, such as, for example, byproducts originating from esterification side reactions.

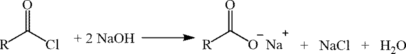

The final step consists of a finishing step, of giving form to the N-acyl derivative formed, and two alternatives are possible.

The first consists in adjusting the value of the pH of the reaction medium obtained to about 7. The N-acyl derivative is isolated as is in solution, without any additional purification, and comprises the acylation salts, the unreacted amino acids and the optional cosolvent. It is thus in a salified form, and more precisely a carboxylate form, in aqueous solution and with a purity generally of less than 50%.

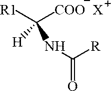

R1 = Side chain of amino acid,
R = Cocoyl chain,
X = Counterion.

The second consists in precipitating the N-acyl derivative by acidifying the reaction mixture to a pH value in the region of 2 and in then performing several filtration and washing operations, concluding with a final drying of the medium obtained. This procedure thus makes it possible to remove all the salts generated during the acylation reaction, the optional acylation cosolvent and all the unreacted amino acids. In this case, the N-acyl derivative is in a non-salified form, with a carboxylic acid function, and a solid form, more particularly a pulverulent form, and with a purity of greater than 80%.

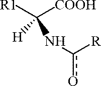

R1 = Side chain of amino acid,
R = Cocoyl chain,
X = Counterion.

The Schotten-Baumann process described above exhibits the advantage of carrying out N-acylation reactions with rapid kinetics, due to the very high reactivity of the acid chlorides toward nucleophilic compounds and functions (for example, the amine function), without a substantial supply of energy, such as, for example, heat energy, in a solvent medium predominantly formed of water, with high yields.

Nowadays, however, formulators of cosmetic products face difficulties regarding the use of powders when it comes to preparing finished products. Furthermore, residual fatty acids, the acyl radical of which contains less than 12 carbon atoms, can generate, when they are present in too large an amount, skin irritation reactions and/or unpleasant odors, incompatible with use in the preparation of cosmetic compositions. For that reason, there is a need to develop a method for the direct synthesis of highly pure liquid salts of salified N-acyl amino acids without precipitation/filtration/drying stages.

The inventors found that it was possible to achieve this result using acylation cosolvents that can also be used as final dilution solvents, it being understood that such cosolvents must also make it possible to control and/or minimize the formation of fatty acids during the acylation step, be poorly reactive to acid chlorides under the reaction conditions, allow for the effective elimination of the salts generated during acylation by simple liquid/liquid decanting at high temperature, and lastly make it possible to obtain a solution of homogeneous N-acyl amino acids with neutral pH.

The French patent application published under the publication number FR 2765105A2 describes a process for preparing a mixture of N-acyl amino acid derivatives using, in particular, an undefined quantity of propylene glycol (or 1,2-propanediol), lauroyl chloride, and indicates a residual lauric acid content in the final product of 15% by mass.

### SUMMARY OF THE INVENTION

That is why, according to a first aspect, a subject of the invention is a composition (C₁) comprising, per 100% of its mass:

(a) - A mass proportion of greater than or equal to 50% by mass and less than or equal to 95% by mass, more particularly greater than or equal to 60% by mass and less than or equal to 80% by mass, of a compound of formula (I) or of a mixture of compounds of formula (I):

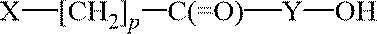

wherein p represents an integer greater than or equal to 5 and less than or equal to 8, Y represents either a divalent radical of formula (IIₐ): 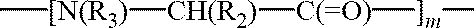
wherein R₃ represents a hydrogen atom or the methyl radical, m represents an integer greater than or equal to 1 and less than or equal to 4 and R₂ represents a hydrogen atom or a radical chosen from methyl, isopropyl, isobutyl, 1-methylpropyl, benzyl and 3-aminopropyl radicals; or a divalent radical of formula (II_{b}): 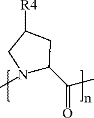
wherein R₄ represents a hydrogen atom or the hydroxyl radical and n represents an integer greater than or equal to 1 and less than or equal to 4; and X represents either the methyl radical or the methylene radical (CH₂═), or the monovalent radical of formula (III): 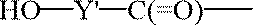
wherein Y′ represents either the divalent radical of formula (IIₐ) as defined previously, or the divalent radical of formula (II_{b}) as defined previously, it being understood that when X represents the radical of formula (III), Y and Y′ are identical;
(b) - A mass proportion of greater than 0% by mass and less than or equal to 15% by mass, more particularly greater than or equal to 5% by mass and less than or equal to 10% by mass, of a diol comprising from three to eight atoms and represented either by formula (IVₐ):

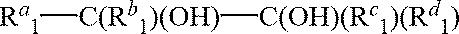

wherein each of the radicals R^{a}₁, R^{b}₁, R^{c}₁ and R^{d}₁ represent, independently of one another, a hydrogen atom or saturated aliphatic radical containing from one to five carbon atoms, or by formula (IV_{b}): 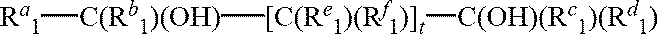
wherein t is equal to one, two or three, each of the radicals R^{a}₁, R^{b}₁, R^{c}₁, R^{d}₁, R^{e}₁ and R^{f}₁ represent, independently of one another, a hydrogen atom or saturated aliphatic radical containing from one to five carbon atoms, it being understood that at least one of the radicals R^{a}₁ or R^{b}₁ and/or at least one of the radicals R^{c}₁ or R^{d}₁ do not represent a hydrogen atom;
(c) - A mass proportion of greater than or equal to 0% by mass and less than or equal to 5% by mass, more particularly greater than or equal to 0.5% by mass and less than or equal to 2.5% by mass, of a compound of formula (V) or of a mixture of compounds of formula (V):

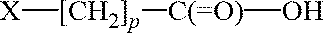

wherein X represents a methyl radical or the vinyl radical (CH₂═CH—), p represents an integer greater than or equal to 5 and less than or equal to 8, or of a mixture of said compounds of formula (V); and
(d) - A mass proportion of greater than 0% by mass and less than 50% by mass, more particularly greater than or equal to 10% by mass and less than or equal to 25% by mass, of water,

it being understood that the pH of said composition is less than or equal to 3, more particularly less than or equal to 2.0.

According to one particular aspect, a subject of the invention is the composition (C₁) as defined above, characterized in that, in the formulae (I) and (V), X represents the methyl radical; according to this particular aspect, in formulae (I) and (V), the monovalent radical X—(CH₂)ₚ—C(═O) more particularly represents a radical chosen from heptanoyl, octanoyl, decanoyl or 10-undecylenoyl radicals. According to this more particular aspect, the composition (C₁) as defined above is most particularly characterized in that, in formula (I), the divalent radical Y represents the divalent radical of formula (IIₐ₁):

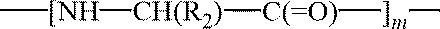

wherein m represents an integer greater than or equal to one and less than or equal to four and R₂ represents a hydrogen atom or a radical chosen from methyl, isopropyl, isobutyl, 1-methylpropyl, benzyl or 3-aminopropyl radicals; for example the divalent radical of formula (II′ₐ₁):

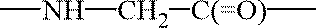

such as the composition (C₁) as defined above, characterized in that, in formulae (I) and (V), the monovalent radical X—(CH₂)ₚ—C(═O)— represents the octanoyl radical.

According to another particular aspect, the composition (C₁) as defined above is characterized in that, in formulae (I) and (V), the monovalent radical X—(CH₂)ₚ—C(═O)— represents the 10-undecylenoyl radical and Y represents the divalent radical of formula (IIₐ₂):

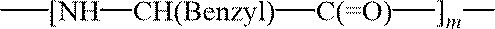

The expression “diol comprising from three to eight atoms and represented either by formula (IVₐ) or by formula (IV_{b})” denotes in particular in the composition (C₁) as defined above 1,2-propanediol, 1,2-butanediol, 1,2-pentanediol, 1,2-hexanediol, 1,2-octanediol, 1,3-butanediol, 2,3-butanediol, 2,3-pentanediol, 2,3-hexanediol, 2,5-hexanediol, or 2-methyl 2,4-pentanediol; and more particularly 1,2-propanediol, 1,2-butanediol, 1,3-butanediol, 1,2-pentanediol, 1,2-hexanediol, or 2-methyl 2,4-pentanediol.

A subject of the invention is also a process for preparing the composition (C₁) as defined previously, characterized in that it comprises the following successive steps:
A step a) during which said diol comprising from three to eight atoms and represented either by formula (IVₐ) or by formula (IV_{b}) is mixed with water to form a mixture (S₁) comprising, per 100% of its mass, from 5% by mass to 70% by mass, and more particularly from 5% by mass to 30% by mass, of said vicinal diol of formula (IVₐ) or of formula (IV_{b}) and from 30% by mass to 95% by mass, and more particularly from 70% by mass to 95% by mass, of water;
A step b) during which a compound of formula (VIₐ):

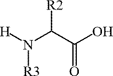

or of formula (VIb):

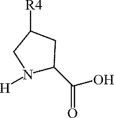

or a mixture of at least one compound of formula (VIₐ) or (VI_{b}) with at least one other compound of formula (VIₐ) or (VI_{b}) is added to said mixture (S₁) in a proportion such that the mixture (M₁) obtained contains, per 100% of its mass, from 5% by mass to 50% by mass, more particularly from 5% by mass to 30% by mass, of said compound of formula (IVₐ) or (IV_{b}) or of said mixture of compounds of formula (VIₐ) and/or (VI_{b}) and from 50% by mass to 95% by mass, more particularly from 50% by mass to 70% by mass, of said mixture (S₁);
A step c) during which sodium hydroxide or potassium hydroxide is added to said mixture (M₁) to form a mixture (M₂) having a pH greater than or equal to 9, more particularly greater than or equal to 9.5;
A step d) during which between 0.7 and 1.0 molar equivalent, more particularly between 0.8 and 0.9 molar equivalent, of an acid chloride of formula (VII):

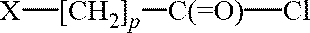

wherein X and p are as defined in formula (I) above, or a mixture of acid chlorides of formula (VII), is poured into said mixture (M₂), while maintaining the pH at a value greater than or equal to 9, more particularly greater than or equal to 9.5, by the joint addition of sodium hydroxide or potassium hydroxide, said step e) resulting in the formation of a mixture (M₃);
A step e) during which said mixture (M₃) is acidified until a pH value of less than or equal to 3, more particularly less than or equal to 2.5, is reached;
A step f) of decanting for the purpose of drawing off the mother liquors;
if necessary or if desired, at least one step g) of washing with brine water to obtain, after decanting, said desired composition.

Step a) of the process as defined above is generally carried out at ambient temperature. According to a particular aspect thereof, said diol comprising from three to eight atoms and represented either by formula (IVₐ) or by formula (IV_{b}) is mixed with water, to form a mixture (S₁) comprising, per 100% of its mass, from 10% by mass to 20% by mass of said vicinal diol of formula (IVₐ) or of formula (IV_{b}) and from 80% by mass to 90% by mass of water;
Step b) of the process as defined above is generally carried out at ambient temperature. According to a particular aspect thereof, said compound of formula (IVₐ) or of formula (IV_{b}), or said mixture of at least one compound of formula (IVₐ) or (IV_{b}) with at least one other compound of formula (IVₐ) or (IV_{b}), is added to said mixture (S₁) in a proportion such that the mixture (M₁) obtained contains, per 100% of its mass, from 10% by mass to 25% by mass of said compound of formula (IVₐ) or (IV_{b}) or of said mixture of compounds of formula (IVₐ) and/or (IV_{b}) and from 75% by mass to 90% by mass of said mixture (S₁).
Step d) of the process as defined above is generally carried out by gradually pouring the acid chloride of formula (VII) or the mixture of acid chlorides of formula (VII) into said mixture (M₂) while maintaining the reaction temperature in a range of between 10° C. and 40° C.; then by leaving the reaction to proceed for approximately thirty minutes at a temperature of between 40° C. and 60° C.
Step f) of the process as defined above is generally carried out by maintaining the reaction temperature in a range of between 40° C. and 60° C.

A subject of the invention is also a process for preparing a cosmetic, dermocosmetic, dermopharmaceutical or pharmaceutical composition for topical use (C) comprising a mass proportion of greater than or equal to 5% by mass and less than or equal to 30% by mass of a compound of formula (I′) or of a mixture of compounds of formula (I′):

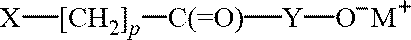

wherein p represents an integer greater than or equal to 5 and less than or equal to 8, M⁺ represents the sodium cation, Y represents either a divalent radical of formula (IIₐ):

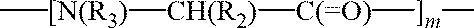

wherein R₃ represents a hydrogen atom or the methyl radical, m represents an integer greater than or equal to 1 and less than or equal to 4 and R₂ represents a hydrogen atom or a radical chosen from methyl, isopropyl, isobutyl, 1-methylpropyl, benzyl and 3-aminopropyl radicals; or a divalent radical of formula (II_{b}):

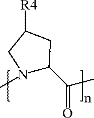

wherein R₄ represents a hydrogen atom or the hydroxyl radical and n represents an integer greater than or equal to 1 and less than or equal to 4; and X represents either the methyl radical or the methylene radical (CH₂═), or the monovalent radical of formula (III):

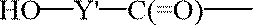

wherein Y′ represents either the divalent radical of formula (IIₐ) as defined above, or the divalent radical of formula (II_{b}) as defined previously, it being understood that when X represents the radical of formula (III), Y and Y′ are identical;

(b) - A mass proportion of greater than 5% by mass and less than or equal to 30% by mass of a diol comprising from three to eight atoms and represented either by formula (IVₐ):

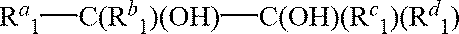

wherein each of the radicals R^{a}₁, R^{b}₁, R^{c}₁ and R^{d}₁ represent, independently of one another, a hydrogen atom or saturated aliphatic radical containing from one to five carbon atoms, or by formula (IV_{b}):

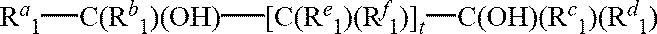

wherein t is equal to one, two or three, each of the radicals R^{a}₁, R^{b}₁, R^{c}₁, R^{d}₁, R^{e}₁ and R^{f}₁ represent, independently of one another, a hydrogen atom or saturated aliphatic radical containing from one to five carbon atoms, it being understood that at least one of the radicals R^{a}₁ or R^{b}₁ and at least one of the radicals R^{c}₁ or R^{d}₁ do not represent a hydrogen atom;

(d) - A mass proportion of greater than 40% by mass and less than 90% by mass of water,
it being understood that the pH of said composition (C) is less than or equal to 9 and greater than or equal to 5.5,
said process being characterized in that it comprises a step h), during which are simultaneously or sequentially added to the composition (C₁) as defined in any one of claims 1 to 7, the necessary amounts of diol of formula (IVa) or of formula (IVb), in aqueous sodium hydroxide solution and in water, until the proportions and pH value as defined above are reached.

The expression “for topical use” used in the definition of said cosmetic, dermocosmetic, dermopharmaceutical or pharmaceutical composition for topical use (C) obtained by the process as defined above means that said composition is employed by application to the skin, the hair, the scalp or the mucous membranes, whether it is a direct application in the case of a cosmetic, dermocosmetic, dermopharmaceutical or pharmaceutical composition or an indirect application, for example in the case of a body hygiene product in the form of a textile or paper wipe, or sanitary products intended to be in contact with the skin or the mucous membranes.

The cosmetic, dermocosmetic, dermopharmaceutical or pharmaceutical composition for topical use (C) obtained by the process as defined above can be packaged in pressurized form in an aerosol device or in a device of “pump-action spray” type, in a device equipped with a perforated wall, for example a grill, or in a device equipped with a ball applicator (known as a “roll-on”). When it is packaged in small bottles, said composition (C) can be applied in the form of fine droplets by means of mechanical pressurization devices or propellent gas devices. Among the propellants that may be combined with composition (C) according to the invention are hydrofluoro compounds, for instance dichlorodifluoromethane, trichlorofluoromethane, difluoroethane, isobutane, butane and propane.

Said composition (C) obtained by the process as defined above may also include excipients and/or active principles commonly used in the field of formulations for topical use, in particular cosmetic, dermocosmetic, pharmaceutical or dermopharmaceutical formulations.

Such excipients are, for example, foaming and/or detergent surfactants, thickening and/or gelling surfactants, thickeners and/or gelling agents, stabilizers, film-forming compounds, solvents and cosolvents, hydrotropic agents, plasticizers, opacifiers, nacreous agents, sequestrants, chelating agents, antioxidants, fragrances, essential oils, preservatives, conditioning agents, particles which provide a visual effect or which are intended for encapsulating active agents, exfoliating particles, texturing agents, optical brighteners.

Such active principles are, for example, intended to provide a treating and/or protective action to the skin or the hair, such as sunscreens, mineral fillers or pigments, insect repellents, deodorants or bleaching agents intended for bleaching bodily hair and the skin.

Among the water-soluble antioxidants that may be included in said composition (C) are, for example, ascorbic acid, glutathione, tartaric acid, oxalic acid and tetrasodium glutamate diacetate.

Among the water-soluble sequestrants that may be included in said composition (C) are, for example, ethylenediaminetetraacetic acid (EDTA) salts, for instance the sodium salt of EDTA, diethylenetriaminepentaacetic acid (DTPA) salts, for instance the sodium salts of DTPA, and acetyl glutamic acid (Dissolvine range).

Among the water-soluble dyes that may be included in said composition (C) are, for example, caramel, Yellow 5, Acid Blue 9/Blue 1, Green 5, Green 3/Fast Green FCF 3, Orange 4, Red 4/Food Red 1, Yellow 6, Acid Red 33/Food Red 12, Red 40, cochineal carmine (CI 15850, CI 75470), Ext. Violet 2, Red 6-7, Ferric Ferrocyanide, Ultramarines, Acid Yellow 3/Yellow 10, Acid Blue 3, Yellow 10.

Among the color-stabilizing water-soluble agents that may be included in said composition (C) are, for example, tris(tetramethylhydroxypiperidinol) citrate, sodium benzotriazolyl butylphenol sulfonate or benzotriazolyl dodecyl p-cresol.

As examples of foaming and/or detergent surfactants optionally present in said composition (C), mention may be made of topically acceptable anionic, cationic, amphoteric or nonionic foaming and/or detergent surfactants commonly used in this field of activity.

Among the foaming and/or detergent anionic surfactants that may be included in said composition (C) are, for example, alkali metal salts, alkaline-earth metal salts, ammonium salts, amine salts, amino alcohol salts of alkyl ether sulfates, of alkyl sulfates, of alkylamido ether sulfates, of alkylarylpolyether sulfates, of monoglyceride sulfates, of alpha-olefin sulfonates, of paraffin sulfonates, of alkyl phosphates, of alkyl ether phosphates, of alkyl sulfonates, of alkylamide sulfonates, of alkylaryl sulfonates, of alkyl carboxylates, of alkylsulfosuccinates, of alkyl ether sulfosuccinates, of alkylamide sulfosuccinates, of alkyl sulfoacetates, of alkyl sarcosinates, of acylisethionates, of N-acyl taurates, of acyl lactylates, of N-acyl amino acid derivatives, of N-acyl peptide derivatives, of N-acyl protein derivatives and of fatty acids.

Among the foaming and/or detergent amphoteric surfactants optionally present in said composition (C), mention may be made of alkylbetaines, alkylamidobetaines, sultaines, alkylamidoalkylsulfobetaines, imidazoline derivatives, phosphobetaines, amphopolyacetates and amphopropionates.

Among the foaming and/or detergent cationic surfactants optionally present in said composition (C), mention may be made particularly of quaternary ammonium derivatives.

Among the foaming and/or detergent nonionic surfactants optionally present in said composition (C), mention may be made more particularly of alkylpolyglycosides containing a linear or branched, saturated or unsaturated aliphatic radical and containing from eight to twelve carbon atoms; castor oil derivatives, polysorbates, coconut kernel amides and N-alkylamines.

As examples of texturing agents optionally present in said composition (C), mention may be made of N-acyl amino acid derivatives, for example lauroyl lysine sold under the name Aminohope™LL, octenyl starch succinate sold under the name Dryflo™, myristyl polyglucoside sold under the name Montanov 14, cellulose fibers, cotton fibers, chitosan fibers, talc, sericite, mica and perlite.

Examples of active principles optionally present in said composition (C) include:
vitamins and derivatives thereof, for example retinol (vitamin A) and esters thereof (for example retinyl palmitate), ascorbic acid (vitamin C) in salt form and esters thereof, sugar derivatives of ascorbic acid (for example ascorbyl glucoside), tocopherol (vitamin E) and esters thereof (for example tocopheryl acetate), vitamin B3 or B10 (niacinamide and derivatives thereof);
compounds having a lightening or depigmenting action on the skin, for example Sepiwhite™ MSH, arbutin, kojic acid, hydroquinone, Vegewhite™, Gatuline™, Synerlight™, Biowhite™, Phytolight™, Dermalight™, Clariskin™, Melaslow™, Dermawhite™, Ethioline, Melarest™, Gigawhite™, Albatine™ and Lumiskin™;
compounds with a calmative action, such as Sepicalm™ S, allantoin and bisabolol;
antiinflammatory agents;
compounds with a moisturizing action, for example diglycerol, triglycerol, urea, hydroxyureas, glyceryl glucoside, diglyceryl glucoside, polyglyceryl glucosides, erythrityl glucoside, sorbityl glucoside, xylityl glucoside, the composition sold under the brand name Aquaxyl™ comprising xylityl glucoside, anhydroxylitol and xylitol;
compounds with a slimming or lipolytic action, such as caffeine or derivatives thereof, Adiposlim™ and Adipoless™;
plant extracts rich in tannins, polyphenols and/or isoflavones, for example grape extracts, pine extracts, wine extracts, olive extracts; soybean extracts, for example Raffermine™; wheat extracts, for example Tensine™ or Gliadine™; terpene-rich plant extracts; freshwater or seawater algal extracts; marine extracts in general such as corals;
compounds with an antimicrobial action or with a purifying action, for example Lipacide™ C8G, Lipacide™UG, Sepicontrol™ A5; Octopirox™ or Sensiva™ SC50;
compounds with an energizing or stimulating property, such as Physiogenyl™, panthenol and derivatives thereof such as Sepicap™ MP;
anti-aging active agents such as Sepilift™ DPHP, Lipacide™PVB, Sepivinol™, Sepivital™, Manoliva™, Phyto-Age™, Timecode™; Survicode™;
anti-photoaging active agents;
active agents which increase the synthesis of extracellular matrix components, for example collagen, elastins and glycosaminoglycans;
active agents acting favorably on chemical cellular communication, such as cytokines, or physical cellular communication, such as integrins;
active agents which create a “heating” sensation on the skin, such as skin capillary circulation activators (for example nicotinic acid derivatives) or products which create a “freshness” sensation on the skin (for example menthol and derivatives thereof);
active agents which improve the skin capillary circulation, for example venotonic agents; draining active agents; decongesting active agents, for example extracts of Ginkgo biloba, ivy, common horse chestnut, bamboo, ruscus, butcher’s broom, Centella asiatica, fucus, rosemary or willow;
active agents which act as skin-tautening agents, for example plant protein hydrolysates, hydrolysates of marine origin, for instance hydrolysates of laminaria extracts, fish cartilage hydrolysates, marine elastin, the product sold by the company SEPPIC under the brand name Sesaflash™, or collagen solutions;
skin tanning or browning agents, for example dihydroxyacetone, isatin, alloxan, ninhydrin, glyceraldehyde, mesotartaric aldehyde, glutaraldehyde or erythrulose.

As examples of deodorants optionally present in said composition (C), mention may be made of alkali metal silicates, zinc salts, such as zinc sulfate, zinc gluconate, zinc chloride or zinc lactate; quaternary ammonium salts, such as cetyltrimethylammonium salts or cetylpyridinium salts; glycerol derivatives, such as glyceryl caprate, glyceryl caprylate or polyglyceryl caprate; 1,2-decanediol, 1,3-propanediol, salicylic acid, sodium bicarbonate, cyclodextrins, metal zeolites, Triclosan™, aluminum bromohydrate, aluminum chlorohydrates, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octachlorohydrate, aluminum sulfate, sodium aluminum lactate, complexes of aluminum chlorohydrate and of glycol, such as the aluminum chlorohydrate and propylene glycol complex, the aluminum dichlorohydrate and propylene glycol complex, the aluminum sesquichlorohydrate and propylene glycol complex, the aluminum chlorohydrate and polyethylene glycol complex, the aluminum dichlorohydrate and polyethylene glycol complex or the aluminum sesquichlorohydrate and polyethylene glycol complex.

As examples of thickeners or gelling agents optionally present in said composition (C), mention may be made of linear or branched or crosslinked polymers of polyelectrolyte type, such as the partially or totally salified acrylic acid homopolymer, the partially or totally salified methacrylic acid homopolymer, the partially or totally salified 2-methyl-[(1-oxo-2-propenyl)amino]-1-propanesulfonic acid (AMPS) homopolymer, copolymers of acrylic acid and of AMPS, copolymers of acrylamide and of AMPS, copolymers of vinylpyrrolidone and of AMPS, copolymers of AMPS and of (2-hydroxyethyl) acrylate, copolymers of AMPS and of (2-hydroxyethyl) methacrylate, copolymers of AMPS and of hydroxyethylacrylamide, copolymers of AMPS and of N,N-dimethylacrylamide, copolymers of AMPS and of tris(hydroxymethyl)acrylamidomethane (THAM), copolymers of acrylic or methacrylic acid and of (2-hydroxyethyl) acrylate, copolymers of acrylic or methacrylic acid and of (2-hydroxyethyl) methacrylate, copolymers of acrylic or methacrylic acid and of hydroxyethylacrylamide, copolymers of acrylic or methacrylic acid and of THAM, copolymers of acrylic or methacrylic acid and of N,N-dimethylacrylamide, terpolymers of acrylic or methacrylic acid, of AMPS and of (2-hydroxyethyl) acrylate, terpolymers of acrylic or methacrylic acid, of AMPS and of (2-hydroxyethyl) methacrylate, terpolymers of acrylic or methacrylic acid, of AMPS and of THAM, terpolymers of acrylic or methacrylic acid, of AMPS and of N,N-dimethylacrylamide, terpolymers of acrylic or methacrylic acid, of AMPS and of acrylamide, copolymers of acrylic acid or of methacrylic acid and of alkyl acrylates, the carbon chain of which comprises between four and thirty carbon atoms and more particularly between ten and thirty carbon atoms, copolymers of AMPS and of alkyl acrylates, the carbon chain of which comprises between four and thirty carbon atoms and more particularly between ten and thirty carbon atoms, linear, branched or crosslinked terpolymers of at least one monomer having a free, partially salified or totally salified strong acid function, with at least one neutral monomer, and at least one monomer of formula (VIII):

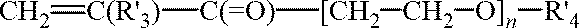

wherein R′₃ represents a hydrogen atom or a methyl radical, R′₄ represents a linear or branched alkyl radical containing from eight to thirty carbon atoms and n represents a number greater than or equal to one and less than or equal to fifty.

The linear or branched or crosslinked polymers of polyelectrolyte type that may be included in said composition (C) may be present in the form of a solution, an aqueous suspension, a water-in-oil emulsion, an oil-in-water emulsion, a powder, for example the products sold under the names Simule™ EG, Simulgel™ EPG, Sepigel™ 305, Simulgel™ 600, Simulgel™ NS, Simulgel™ INS 100, Simulgel™ FL, Simulgel™ A, Simulgel™ SMS 88, Sepinov™ EMT 10, Sepiplus™ 400, Sepiplus™ 265, Sepiplus™ S, Sepimax™ Zen, Aristoflex™ AVC, Aristoflex™ AVS, Novemer™ EC-1, Novemer™ EC 2, Aristoflex™ HMB, Cosmedia™ SP, Flocare™ ET 25, Flocare™ ET 75, Flocare™ ET 26, Flocare™ ET 30, Flocare™ ET 58, Flocare™ PSD 30, Viscolam™ AT 64 and Viscolam™ AT 100; polysaccharides constituted solely of saccharides, such as glucans or glucose homopolymers, glucomannoglucans, xyloglucans, galactomannans in which the degree of substitution (DS) of the D-galactose units on the D-mannose main chain is between 0 and 1 and more particularly between 1 and 0.25, for instance galactomannans originating from cassia gum (DS = ⅕), from locust bean gum (DS = ¼), from tara gum (DS = ⅓), from guar gum (DS = ½) or from fenugreek gum (DS = 1); polysaccharides constituted of saccharide derivatives, such as galactan sulfates and more particularly carrageenans and agar, uronans and more particularly algins, alginates and pectins, heteropolymers of saccharides and of uronic acids and more particularly xanthan gum, gellan gum, gum arabic exudates and karaya gum exudates, glucosaminoglycans; cellulose, cellulose derivatives such as methyl-cellulose, ethyl-cellulose, hydroxypropyl cellulose, silicates, starch, hydrophilic starch derivatives, polyurethanes.

As examples of oils that may be present in said composition (C), mention may be made of mineral oils such as liquid paraffin, liquid petroleum jelly, isoparaffins or white mineral oils; oils of animal origin, such as squalene or squalane; plant oils, such as phytosqualane, sweet almond oil, coconut kernel oil, castor oil, jojoba oil, olive oil, rapeseed oil, peanut oil, sunflower oil, wheat germ oil, corn germ oil, soybean oil, cotton oil, alfalfa oil, poppy oil, pumpkin oil, evening primrose oil, millet oil, barley oil, rye oil, safflower oil, candlenut oil, passionflower oil, hazelnut oil, palm oil, shea butter, apricot kernel oil, coriander seed oil, beechnut oil, beauty-leaf oil, sisymbrium oil, avocado oil, calendula oil, oils derived from flowers or vegetables, ethoxylated plant oils; synthetic oils, for instance fatty acid esters such as butyl myristate, propyl myristate, isopropyl myristate, cetyl myristate, isopropyl palmitate, octyl palmitate, butyl stearate, hexadecyl stearate, isopropyl stearate, octyl stearate, isocetyl stearate, dodecyl oleate, hexyl laurate, propylene glycol dicaprylate, esters derived from lanolic acid, such as isopropyl lanolate, isocetyl lanolate, fatty acid monoglycerides, diglycerides and triglycerides, for instance glyceryl triheptanoate, alkylbenzoates, hydrogenated oils, poly(alpha-olefins), polyolefins such as poly(isobutane), synthetic isoalkanes, for instance isohexadecane, isododecane, perfluorinated oils; silicone oils, for instance dimethylpolysiloxanes, methylphenylpolysiloxanes, silicones modified with amines, silicones modified with fatty acids, silicones modified with alcohols, silicones modified with alcohols and fatty acids, silicones modified with polyether groups, epoxy-modified silicones, silicones modified with fluoro groups, cyclic silicones and silicones modified with alkyl groups. In the present patent application, the term “oils” refers to compounds and/or mixtures of compounds which are water-insoluble, and which have a liquid appearance at a temperature of 25° C.

As examples of waxes optionally present in said composition (C), mention may be made of beeswax, carnauba wax, candelilla wax, ouricury wax, Japan wax, cork fiber wax, sugarcane wax, paraffin waxes, lignite waxes, microcrystalline waxes, lanolin wax; ozokerite; polyethylene wax; silicone waxes; plant waxes; fatty alcohols and fatty acids that are solid at ambient temperature; glycerides that are solid at ambient temperature. In the present patent application, the term “waxes” refers to compounds and/or mixtures of compounds which are water-insoluble, and which have a solid appearance at a temperature of greater than or equal to 45° C.

As examples of emulsifying nonionic surfactants that may be included in said composition (C), mention may be made of fatty acid esters of sorbitol, for instance the products sold under the names Montane™ 40, Montane™ 60, Montane™ 70, Montane™ 80 and Montane™ 85; compositions comprising glyceryl stearate and stearic acid ethoxylated with between 5 mol and 150 mol of ethylene oxide, for instance the composition comprising stearic acid ethoxylated with 135 mol of ethylene oxide and glyceryl stearate sold under the name Simulsol™ 165; mannitan esters; ethoxylated mannitan esters; sucrose esters; methyl glucoside esters; alkyl polyglycosides containing a linear or branched, saturated or unsaturated aliphatic radical containing from 14 to 36 carbon atoms, for instance tetradecyl polyglucoside, hexadecyl polyglucoside, octadecyl polyglucoside, hexadecyl polyxyloside, octadecyl polyxyloside, eicosyl polyglucoside, dodecosyl polyglucoside, (2-octyldodecyl) polyxyloside, (12-hydroxystearyl) polyglucoside; compositions of linear or branched, saturated or unsaturated fatty alcohols containing from 14 to 36 carbon atoms and of alkyl polyglycosides as described previously, for example the compositions sold under the brand names Montanov™ 68, Montanov™ 14, Montanov™ 82, Montanov™ 202, Montanov™ S, Montanov™ WO18, Montanov™ L, Fluidanov™ 20X and Easynov™.

As examples of agents for protecting against ultraviolet radiation from the sun optionally present in said composition (C), pigments, organic sunscreens and inorganic sunscreens are denoted.

As pigments used as an agent for protecting against ultraviolet radiation from the sun optionally present in said composition (C), there are for example titanium dioxide, brown iron oxides, yellow iron oxides, black iron oxides or red iron oxides, or else white or colored nacreous pigments such as titanium mica.

As organic sunscreens used as an agent for protecting against ultraviolet radiation from the sun optionally present in said composition (C), there are for example:
those of the family of benzoic acid derivatives, such as para-aminobenzoic acids (PABAs), notably monoglyceryl esters of PABA, ethyl esters of N,N-propoxy PABA, ethyl esters of N,N-diethoxy PABA, ethyl esters of N,N-dimethyl PABA, methyl esters of N,N-dimethyl PABA, butyl esters of N,N-dimethyl PABA;
those of the family of anthranilic acid derivatives, such as homomenthyl-N-acetyl anthranilate;
those of the family of salicylic acid derivatives, such as amyl salicylate, homomenthyl salicylate, ethylhexyl salicylate, phenyl salicylate, benzyl salicylate, p-isopropanolphenyl salicylate;
those of the family of cinnamic acid derivatives, such as ethylhexyl cinnamate, ethyl-4-isopropyl cinnamate, methyl-2,5-diisopropyl cinnamate, p-methoxypropyl cinnamate, p-methoxyisopropyl cinnamate, p-methoxyisoamyl cinnamate, p-methoxyoctyl cinnamate (p-methoxy 2-ethylhexyl cinnamate), p-methoxy 2-ethoxyethyl cinnamate, p-methoxycyclohexyl cinnamate, ethyl-α-cyano-β-phenyl cinnamate, 2-ethylhexyl-α-cyano-β-phenyl cinnamate, glyceryl di-para-methoxy mono-2-ethylhexanoyl cinnamate;
those of the family of benzophenone derivatives, such as 2,4-dihydroxybenzophenone, 2,2′-dihydroxy-4-methoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl 4′-phenylbenzophenone-2-carboxylate, 2-hydroxy 4-n-octyloxybenzophenone, 4-hydroxy 3-carboxybenzophenone; 3-(4′-methylbenzylidene)-d,l-camphor, 3-(benzylidene)-d,l-camphor, benzalkonium methosulfate camphor; urocanic acid, ethyl urocanate;
those of the family of sulfonic acid derivatives, such as 2-phenylbenzimidazole-5-sulfonic acid and its salts; the family of triazine derivatives, such as hydroxyphenyl triazine, ethylhexyloxyhydroxyphenyl-4-methoxyphenyltriazine, 2,4,6-trianilino-(p-carbo-2′-ethylhexyl-1′-oxy)-1,3,5-triazine, the 4,4-((6-(((1,1-dimethylethyl)amino)carbonyl)phenyl)amino)-1,3,5-triazine-2,4-diyl diimino) bis(2-ethylhexyl) ester of benzoic acid, 2-phenyl-5-methylbenzoxazole, 2,2′-hydroxy-5-methylphenylbenzotriazole, 2-(2′-hydroxy-5′-t-octylphenyl)benzotriazole, 2-(2′-hydroxy-5′-methyphenyl)benzotriazole; dibenzazine; dianisoylmethane, 4-methoxy-4″-t-butylbenzoylmethane; 5-(3,3-dimethyl-2-norbornylidene)-3-pentan-2-one; 2-(4-diethylamino-2-hydroxybenzoyl)benzoic acid hexyl ester, 2,4-bis{[4-(2-ethylhexyloxy)-2-hydroxy]phenyl}-6-(4-methoxyphenyl)-1,3,5-triazine, 2,4,6-tris[4-(2-ethylhexyloxycarbonyl)anilino]-1,3,5-triazine, 2-ethylhexyl dimethoxybenzylidene dioxoimidazolidine propionate, the family of diphenylacrylate derivatives, such as 2-ethylhexyl-2-cyano-3,3-diphenyl-2-propenoate or ethyl-2-cyano-3,3-diphenyl-2-propenoate;
those of the family of polysiloxanes, such as benzylidene siloxane malonate.

As inorganic sunscreens used as an agent for protecting against ultraviolet radiation from the sun optionally present in said composition (C), there are for example titanium oxides, zinc oxides, cerium oxide, zirconium oxide, yellow, red or black iron oxides, chromium oxides. These mineral sunblocks may or may not be micronized, may or may not have undergone surface treatments and may optionally be present in the form of aqueous or oily predispersions.

The examples that follow illustrate the invention without, however, limiting it.

### DESCRIPTION OF THE PREFERRED EMBODIMENTS

### Preparation of Compositions (C₁) According to the Invention

The common procedure is as follows:
Introducing, at ambient temperature and with mechanical stirring, 20% by mass of glycocoll (glycine) into 80% by mass of a mixture of water/diol (85/15 by mass),
Adding 30% sodium hydroxide to achieve a pH of approx. 10,
Gradually pouring in octanoyl chloride (0.9 molar equivalent), while maintaining the pH at approximately 10 with 30% sodium hydroxide, at a temperature of 18 to 36° C.,
Breaking with 37% hydrochloric acid qs to achieve a pH of approx. 2,
Drawing off the mother liquors at 60° C.,
Washing with 5% brine water,
Decanting and drawing off at 65° C.

The features of the compositions (C₁) are collated in table 1 below:

### Preparation of a Comparative Composition (C₂₁)

Introducing, at ambient temperature and with mechanical stirring, 20% by mass of glycocoll (glycine) into 80% by mass of a mixture of water/diol (85/15 by mass),
Adding 30% sodium hydroxide to achieve a pH of approx. 10,
Gradually pouring in lauroyl chloride (0.9 molar equivalent), while maintaining the pH at approximately 10 with 30% sodium hydroxide, at a temperature of 18 to 36° C.,
Breaking with 37% hydrochloric acid qs to achieve a pH of approx. 2,
Drawing off the mother liquors at 60° C.,
Washing with 5% brine water,
Decanting and drawing off at 65° C.

The features of the composition (C₂₁) thus obtained are collated in table 2 below.

### Preparation of a Comparative Composition (C₂₂)

Introducing, at ambient temperature and with mechanical stirring, 20% by mass of a mixture of glycine, L-aspartic acid, L-glutamic acid and L-alanine, in the mass proportions of glycine/L-aspartic acid/L-glutamic acid/L-alanine of 10%/35%/45%/10% per 100% by mass of said mixture of amino acids, into 80% by mass of a mixture of water/diol (85/15 by mass),
Adding 30% sodium hydroxide to achieve a pH of approx. 10,
Gradually pouring in lauroyl chloride (0.9 molar equivalent), while maintaining the pH at approximately 10 with 30% sodium hydroxide, at a temperature of 18 to 36° C.,
Breaking with 37% hydrochloric acid qs to achieve a pH of approx. 2,
Drawing off the mother liquors at 60° C.,
Washing with 5% brine water,
Decanting and drawing off at 65° C.
The features of the composition (C₂₂) thus obtained are collated in table 2 below.

TABLE 1

| | (C_{1A}) | (C_{1B}) | (C_{1C}) |
|---|---|---|---|
| Diol (IVₐ) or (IV_{b}) | 2-methyl-2,4- pentanediol | 1,2-propanediol | 1,3-propanediol |
| Content of diol (IVₐ) or (IV_{b}) | 8.9% by mass | 1.2% by mass | 1.1% by mass |
| Content of octanoic acid (V) | 0.8% by mass | 2.2% by mass | 3.9% by mass |
| Water content | 19.0% by mass | 19.8% by mass | 16.0% by mass |
| Content of N-octanoyl glycine (I) | 71.3% by mass | 76.8% by mass | 78.5% by mass |
| Presence of by-products | No | No | 0.5% by mass |
| pH of the composition | pH = 1.8 | pH = 2.0 | pH = 1.9 |
| | (C_{1D}) | (C_{1E}) | (C_{1F}) |
| Diol (IVₐ) or (IV_{b}) | 1,2-butanediol | 1,3-butanediol | 1,4-butanediol |
| Content of diol (IVₐ) or (IV_{b}) | 8.1% by mass | 3.8% by mass | 0.9% by mass |
| Content of octanoic acid (V) | 0.6% by mass | 2.4% by mass | 3.0% by mass |
| Water content | 19.1% by mass | 20.2% by mass | 23.0% by mass |
| Content of N-octanoyl glycine (I) | 72.2% by mass | 73.6% by mass | 71.9% by mass |
| Presence of by-products | No | No | 1.2% by mass |
| pH of the composition | pH = 1.6 | pH = 1.5 | pH = 1.7 |
| Content of diol (IVₐ) or (IV_{b}) | 6.6% by mass | 2.2% by mass | 10.1% by mass |
| Content of octanoic acid (V) | 0.8% by mass | 2.9% by mass | 0.6% by mass |
| Water content | 19.0% by mass | 18.5% by mass | 16.2% by mass |
| Content of N-octanoyl glycine (I) | 73.6% by mass | 74.6% by mass | 73.1% by mass |
| Presence of by-products | No | 1.8% by mass | No |
| pH of the composition | pH = 1.8 | pH = 2.1 | pH = 2.2 |

TABLE 2

| | (C₂₁) | (C₂₂) |
|---|---|---|
| Diol (IVₐ) or (IV_{b}) | 1,2-propanediol | 1,2-propanediol |
| Content of diol (IVₐ) or (IV_{b}) | 0.9% by mass | 1.9% by mass |
| Content of lauric acid (V) | 4.1% by mass | 12.9% by mass |
| Water content | 11.0% by mass | 27.5% by mass |
| Content of N-lauroyl glycine (I) | 83.2% by mass | 11.9% by mass |
| Content of N-lauroyl alanine (I) | - | 11.0% by mass |
| Content of N-lauroyl aspartic acid (I) | - | 12.2% by mass |
| Content of N-lauroyl glutamic acid (I) | - | 20.4% by mass |
| Presence of by-products | 0.8% by mass | 2.2% by mass |
| pH of the composition | pH = 2.1 | pH = 2.2 |

Analysis of the compositions (C_{1A}), (C_{1B}) and (C_{1D}), (C_{1E}) and (C_{1G}), which are subjects of the present invention, reveals that they do not contain by-products, unlike the compositions (C_{1C}), (C_{1F}), (C₂₁) and (C₂₂), and that their fatty acid concentration is lower than those of the compositions (C_{1C}), (C_{1H}), (C_{1F}), (C₂₁) and (C₂₂).

1. A method of making a composition (C₁) comprising, per 100% of its mass:
(a) - A mass proportion of greater than or equal to 50% by mass and less than or equal to 95% by mass of a compound of formula (I) or of a mixture of compounds of formula (I): 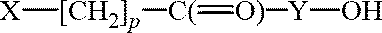
wherein p represents an integer greater than or equal to 5 and less than or equal to 8, Y represents either a divalent radical of formula (IIₐ): 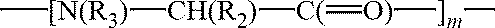
wherein R₃ represents a hydrogen atom or the methyl radical, m represents an integer greater than or equal to 1 and less than or equal to 4 and R₂ represents a hydrogen atom or a radical chosen from methyl, isopropyl, isobutyl, 1-methylpropyl, benzyl and 3-aminopropyl radicals; or a divalent radical of formula (II_{b}): 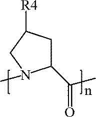
wherein R₄ represents a hydrogen atom or the hydroxyl radical and n represents an integer greater than or equal to 1 and less than or equal to 4; and X represents either the methyl radical or the methylidene radical (CH₂═), or the monovalent radical of formula (III):. 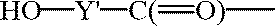
wherein Y′ represents either the divalent radical of formula (IIa) as defined previously, or the divalent radical of formula (IIb) as defined previously, wherein when X represents the radical of formula (III), Y and Y′ are identical;.
(b) - A mass proportion of greater than 0% by mass and less than or equal to 15% by mass of a diol comprising from three to eight atoms and represented either by formula (IVₐ):. 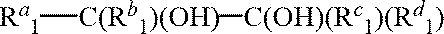
wherein each of the radicals R^{a}₁, R^{b}₁, R^{c}₁ and R^{d}₁ represent, independently of one another, a hydrogen atom or saturated aliphatic radical containing from one to five carbon atoms, or by formula (IV_{b}):. 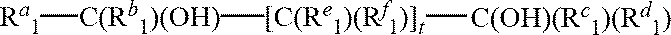
wherein t is equal to one, two or three, each of the radicals R^{a}₁, R^{b}₁, R^{c}₁, R^{d}₁, R^{e}₁ and R^{f}₁ represent,. independently of one another, a hydrogen atom or saturated aliphatic radical containing from one
to five carbon atoms, wherein at least one of the radicals R^{a}₁ or R^{b}₁ and/or at least one of the radicals R^{c}₁ or R^{d}₁ do not represent a hydrogen atom;.
(c) - A mass proportion of greater than or equal to 0% by mass and less than or equal to 5% by mass of a compound of formula (V) or of a mixture of compounds of formula (V): 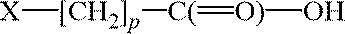
wherein X represents a methyl radical or the vinyl radical (CH₂═CH—), p represents an integer greater than or equal to 5 and less than or equal to 8, or of a mixture of said compounds of formula (V); and.
(d) - A mass proportion of greater than 0% by mass and less than 50% by mass of water,.
wherein the pH of said composition is less than or equal to 3;
said method comprising:
a step i) during which said diol comprising from three to eight atoms and represented either by formula (IVₐ) or by formula (IV_{b}) is mixed with water to form a mixture (S₁) comprising, per 100% of its mass, from 5% by mass to 70% by mass of said diol of formula (IVₐ) or of formula (IV_{b}) and from 30% by mass to 95% by mass of water;
a step ii) during which a compound of formula (VIₐ): 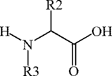
or of formula (VI_{b}): 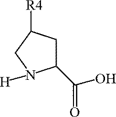
.
or a mixture of at least one compound of formula (VIₐ) or (VI_{b}) with at least one other compound of formula (VIₐ) or (VI_{b}) is added to said mixture (S₁) in a proportion such that the mixture (M₁) obtained contains, per 100% of mass, from 5% by mass to 50% by mass of said compound of formula (VIₐ) or (VI_{b}) or of said mixture of compounds of formula (VIₐ) and/or (VI_{b}) and from 50% by mass to 95% by mass of said mixture (S₁);
a step iii) during which sodium hydroxide or potassium hydroxide is added to said mixture (M₁) to form a mixture (M₂) having a pH greater than or equal to 9;
a step iv) during which an acid chloride of formula (VII): 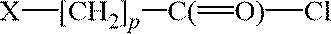
wherein X and p are as defined in formula (I) above, or a mixture of acid chlorides of formula (VII), is poured into said mixture (M₂), while maintaining the pH at a value greater than or equal to 9 by the joint addition of sodium hydroxide or potassium hydroxide, said step e) resulting in the formation of a mixture (M₃);
a step v) during which said mixture (M₃) is acidified until a pH value of less than or equal to 3 is reached;
a step vi) of decanting for the purpose of drawing off the mother liquors; and
optionally at least one step vii) of washing with brine water to obtain, after decanting, said composition
.

2. The method according to claim 1, wherein in formulae (I) and (V), X represents the methyl radical.

3. The method according to claim 1, wherein, in formula (I), the divalent radical Y represents the divalent radical of formula (IIₐ₁): 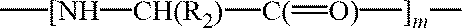
wherein m represents an integer greater than or equal to one and less than or equal to four and R₂ represents a hydrogen atom or a radical chosen from methyl, isopropyl, isobutyl, 1-methylpropyl, benzyl or 3-aminopropyl radicals.

4. The method according to claim 3, wherein, in formula (I), the divalent radical Y represents the divalent radical of formula (II′ₐ₁): 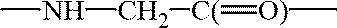
.

5. The method according to claim 4, wherein, in formulae (I) and (V), the monovalent radical X—(CH₂)ₚ—C(═O)— represents the octanoyl radical.

6. The method according to claim 2, wherein, in formulae (I) and (V), the monovalent radical X—(CH₂)ₚ—C(═O)— represents the 10-undecylenoyl radical and Y represents the divalent radical of formula (IIₐ₂): 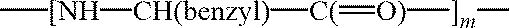
.

7. The method according to claim 1, wherein said diol comprising from three to eight atoms and represented either by formula (IVa) or by formula (IVb) is selected from the group consisting of 1,2-propanediol, 1,2-butanediol, 1,3-butanediol, 1,2-pentanediol, 1,2-hexanediol, 1,2-octanediol, 2,3-butanediol, 2,3-pentanediol, 2,3-hexanediol, 2,5-hexanediol and 2-methyl-2,4-pentanediol.

8. The method according to claim 7, wherein said diol comprising from three to eight atoms and represented either by formula (IVₐ) or by formula (IV_{b}) is chosen from 1,2-propanediol, 1,2-butanediol, 1,3-butanediol, 1,2-pentanediol, 1,2-hexanediol or 2-methyl-2,4-pentanediol.

9. A method of making a cosmetic, dermocosmetic, dermopharmaceutical or pharmaceutical composition for topical use (C) comprising a mass proportion of greater than or equal to 5% by mass and less than or equal to 30% by mass of a compound of formula (I′) or of a mixture of compounds of formula (I′): 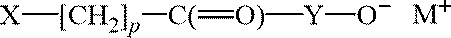
wherein X represents a methyl radical or the vinyl radical (CH₂═CH—), p represents an integer greater than or equal to 5 and less than or equal to 12, M⁺ represents the sodium cation, Y represents either a divalent radical of formula (IIₐ): 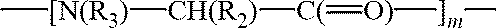
wherein R₃ represents a hydrogen atom or the methyl radical, m represents an integer greater than or equal to 1 and less than or equal to 4 and R₂ represents a hydrogen atom or a radical chosen from methyl, isopropyl, isobutyl, 1-methylpropyl, benzyl and 3-aminopropyl radicals; or a divalent radical of formula (II_{b}): 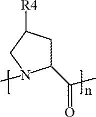
wherein R₄ represents a hydrogen atom or the hydroxyl radical and n represents an integer greater than or equal to 1 and less than or equal to 4; and X represents either the methyl radical or the methylene radical (CH₂═), or the monovalent radical of formula (III): 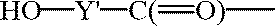
wherein Y′ represents either the divalent radical of formula (IIₐ) as defined above, or the divalent radical of formula (IIb) as defined above, wherein, when X represents the radical of formula (III), Y and Y′ are identical;
(b) - A mass proportion of greater than 5% by mass and less than or equal to 30% by mass of a diol comprising from three to eight atoms and represented either by formula (IVₐ): 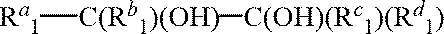
wherein each of the radicals R^{a}₁, R^{b}₁, R^{c}₁ and R^{d}₁ represent, independently of one another, a hydrogen atom or saturated aliphatic radical containing from one to five carbon atoms, or by formula (IV_{b}): 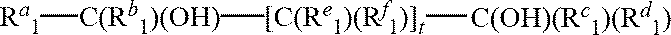
wherein t is equal to one, two or three, each of the radicals R^{a}₁, R^{b}₁, R^{c}₁, R^{d}₁, R^{e}₁ and R^{f}₁ represent, independently of one another, a hydrogen atom or saturated aliphatic radical containing from one to five carbon atoms, it being understood that at least one of the radicals R^{a}₁ or R^{b}₁ and at least one of the radicals R^{c}₁ or R^{d}₁ do not represent a hydrogen atom;
(d) - A mass proportion of greater than 40% by mass and less than 90% by mass of water,
it being understood that the pH of said composition (C) is less than or equal to 9 and greater than or equal to 5.5,
said method comprising a step viii), during which are simultaneously or sequentially added to the composition (C₁) as defined in claim 1, the necessary amounts of diol of formula (IVa) or of formula (Vb), in aqueous sodium hydroxide solution and in water, until the proportions and pH value as defined above are reached
.

10. The method according to claim 9, wherein in formulae (I) and (V), X represents the methyl radical.

11. The method according to claim 9, wherein, in formula (I), the divalent radical Y represents the divalent radical of formula (IIₐ₁): 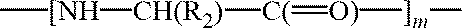
wherein m represents an integer greater than or equal to one and less than or equal to four and R₂ represents a hydrogen atom or a radical chosen from methyl, isopropyl, isobutyl, 1-methylpropyl, benzyl or 3-aminopropyl radicals
.

12. The method according to claim 11, wherein, in formula (I), the divalent radical Y represents the divalent radical of formula (IIₐ₁): 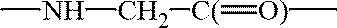
.

13. The method according to claim 12, wherein, in formulae (I) and (V), the monovalent radical X—(CH₂)ₚ—C(═O)— represents the octanoyl radical.

14. The method according to claim 10, wherein, in formulae (I) and (V), the monovalent radical X—(CH₂)ₚ—C(═O)— represents the 10-undecylenoyl radical and Y represents the divalent radical of formula (IIₐ₂): 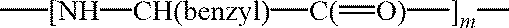
.

15. The method according to claim 9, wherein said diol comprising from three to eight atoms and represented either by formula (IVa) or by formula (IVb) is selected from the group consisting of 1,2-propanediol, 1,2-butanediol, 1,3-butanediol, 1,2-pentanediol, 1,2-hexanediol, 1,2-octanediol, 2,3-butanediol, 2,3-pentanediol, 2,3-hexanediol, 2,5-hexanediol and 2-methyl-2,4-pentanediol.

16. The method according to claim 15, wherein said diol comprising from three to eight atoms and represented either by formula (IVₐ) or by formula (IV_{b}) is chosen from 1,2-propanediol, 1,2-butanediol, 1,3-butanediol, 1,2-pentanediol, 1,2-hexanediol or 2-methyl-2,4-pentanediol.
